(19) United States
(12) Patent Application Publication
(10) Pub. No.: US 2026/0069324 A1
(43) Pub. Date: Mar. 12, 2026

(54) DEVICE AND METHOD FOR PRODUCING AN INTRAMEDULLARY ROD SPACER

(71) Applicant: Heraeus Medical GmbH, Wehrheim (DE)

(72) Inventors: Sebastian VOGT, Wehrheim (DE); Thomas KLUGE, Wehrheim (DE)

(21) Appl. No.: 19/321,954

(22) Filed: Sep. 8, 2025

(30) Foreign Application Priority Data
Sep. 11, 2024 (EP) 24199681.8

(51) Int. Cl. A61B 17/72; A61B 17/00

(52) U.S. Cl. CPC A61B 17/72; A61B 2017/00477; A61B 2017/00526; A61B 2017/00991

(57) ABSTRACT

The present invention relates to a device for producing an intramedullary rod spacer, to a use of a casting mold, and to a method for producing an intramedullary rod spacer. A device for producing an intramedullary rod spacer comprises a first tube, a second tube and a flexible connecting piece for fluidically connecting the first tube to the second tube, wherein the first tube and the second tube can be positioned at different angles relative to one another. In this way, intramedullary rod spacers can be produced intraoperatively and with increased precision.

### CROSS-REFERENCE TO RELATED APPLICATION

This application claims priority pursuant to 35 U.S. C. 119(a) to European Patent Office Application No. 24199681.8, filed Sep. 11, 2024, which application is incorporated herein by reference in its entirety.

### FIELD OF THE INVENTION

The invention relates to a device for producing an intramedullary rod spacer, to a use of a casting mold, and to a method for producing an intramedullary rod spacer.

### BACKGROUND

Intramedullary rods are used to treat bone fractures, for example in trauma surgery. This occurs in the case of fractures of long bones, such as the thigh bone (femur), shin bone (tibia) or upper arm bone (humerus). Intramedullary rods are typically driven lengthwise into the bone cavity and allow new bone tissue to form there. After such treatment, infections of the surrounding bone and/or soft tissue can occur, for example due to bacteria such as Staphylococcus aureus or Staphylococcus epidermidis. This is particularly the case with open fractures and requires surgical treatment in which the intramedullary rod is removed and infected tissue is removed (debridement). In addition, systemic antibiotics and/or local anti-infectives are used to specifically combat the infection.

It is known to provide intramedullary rods with an antibiotic-containing coating made of polymethylmethacrylate bone cement (N. Walter et al.: “Individual and commercially available antimicrobial coatings for intramedullary rods for the treatment of infected long bone non-unions - a systematic review”; Injury 2022; DOI: 10.1016/j. injury.2022.05.008). Antibiotics from the bone cement are dissolved from the coating by aqueous body fluids, such as wound secretions and blood, leading to locally high antibiotic concentrations. In combination with systemic antibiotics, the germs that may remain after debridement can be reduced. However, it is problematic to achieve a uniform coating thickness. Intramedullary rods with a coating that is too thick cannot be implanted into previously debrided long bones or can only be implanted with difficulty.

It is desirable if, after surgical repair and before reinsertion of an intramedullary rod, temporary mechanical stabilization of the medullary cavity is possible and if anti-infectives could also be delivered locally in order to locally suppress microbial germs remaining in the debrided tissue. For this purpose, it is possible to manually form rods made of polymethylmethacrylate bone cement intraoperatively, to partially replicate the previously explanted intramedullary rods. In this case, too, an antibiotic for local release can be provided. However, the fit of manually shaped rods is fundamentally problematic. Manual shaping is not suitable for adequately replicating an intramedullary rod, especially in the case of curved and/or larger rods. For this reason, the results are often unsatisfactory.

### SUMMARY

The above-mentioned features can be combined as desired with the different aspects of the invention.

The object of the invention is to produce intramedullary rod spacers in an improved manner.

The object is achieved by the device according to claim 1, as well as by the use and by the method according to the independent claims. Advantageous embodiments are specified in the dependent claims.

The object is achieved by a device for producing an intramedullary rod spacer. The device comprises a first tube, a second tube, and a flexible connecting piece for fluidically connecting the first tube to the second tube. The first tube and the second tube can be positioned at different angles relative to one another.

The device allows intramedullary rod spacers (hereinafter also referred to as spacers) to be produced in a simple and defined manner. An intramedullary rod spacer can be used in place of the removed intramedullary rod to provide temporary mechanical stabilization of the medullary canal after surgical repair and before reinsertion of an intramedullary rod.

An intramedullary rod spacer can be made from a casting material, such as bone cement. An intramedullary rod spacer can contain one or more active ingredients, such as anti-infectives, for example antibiotics, for local delivery. Active ingredients can be added to the casting material to be incorporated into the intramedullary rod spacer. The device serves as a casting mold. The casting material can be filled into the device and cured to form an intramedullary rod spacer. The cured intramedullary rod spacer can then be removed from the device. In particular, the individual tubes and the connecting piece can be removed individually from the intramedullary rod spacer. Non-destructive removal may be possible in this case-for example, for one or both tubes and/or the connecting piece-and/or separation may occur with destruction of the relevant casting mold-for example the connecting piece.

Depending on the intended implantation site, intramedullary rods can be designed as straight or curved hollow bodies. The positioning of the first tube and the second tube at different angles relative to one another makes it possible to replicate the curvature of the explanted intramedullary rod, if present. Depending on requirements, straight or arbitrarily curved intramedullary rod spacers can be produced. In this way, the removed intramedullary rod can also be reconstructed intraoperatively in order to fit the specified position in the medullary canal as closely as possible. Intraoperative production makes it possible to introduce active ingredients that are precisely tailored to the germs present, such as anti-infectives, for example antibiotics, into the spacer.

A tube is an elongated hollow body the length of which is at least 3 times greater than its external cross-section or diameter. A tube defines an elongated cavity in its interior. An inner and/or outer cross-section of the tube can be circular, but in principle different cross-sections, such as oval, square, rectangular or other, regular polygonal, cross-sections are also possible. Usually, the inner and outer cross-sections correspond to one another. The wall thickness can be constant in the axial direction and/or in the circumferential direction.

A cavity in the first tube and/or the second tube may be conical in shape. If there is a circular cross-section, the inner diameter can increase continuously along the axial direction. Conical tubes are easier to demold. In other words, it is easier to remove the produced intramedullary rod spacer from the associated tube. In some embodiments, the tube does not contain any steps in its interior. A conical tube has an angle of at most 7°, the angle being measured between the longitudinal axis of the tube and the inner wall of the tube.

The first tube is in particular a distal tube, i.e., a tube which is designed to produce a distal portion of an intramedullary rod spacer. The second tube is a proximal tube, i.e. a tube which is designed to produce a proximal portion of an intramedullary rod spacer. In particular, the first tube is longer than the second tube.

The connecting piece serves to connect the first tube to the second tube and/or to create a connecting part between the first portion and the second portion of the intramedullary rod spacer. A fluidic connection is a connection in which the cavity of the first tube is connected to the cavity of the second tube. In particular, the connection is made over at least essentially the entire cross-section of the first tube and/or the second tube. The connecting piece is, in particular, tubular and can be hollow cylindrical and/or conical (in the shape of a hollow truncated cone). If there is a conical connecting piece, intramedullary rod spacers can be produced with portions of different diameters. This may be desirable depending on the patient's anatomy.

In particular, one end of the first tube is connected to one side of the connecting piece and one end of the second tube is connected to the other side of the connecting piece. In particular, the connection is made in such a way that in the connected state there is a connected tube with two ends and a continuous cavity.

The connecting piece can be connected and/or can be connectable to the first tube and/or the second tube. If the connecting piece can be connected to the first tube and/or the second tube, the components can be provided separately and connected by the user. The device can therefore also be present as a system in a disassembled state. Regardless of the connected or separate state upon delivery, the connection between the connecting piece and each tube is preferably detachable, manually detachable, in order to simplify demolding after the casting material has been cured.

The connecting piece is so flexible that an angle of 40° can be set between the first tube and the second tube and thus between the opposite sides of the connecting piece. The angle is measured between the longitudinal axes.

In particular, positioning at a specific angle can be achieved by relative rotation of the first tube with respect to the second tube.

In particular, the first tube and/or the second tube is straight. However, it is also possible that the first tube and/or the second tube is curved. For example, there may be a bend near the connecting piece and/or a straight tube portion may be present in a region further away from the connecting piece.

The adaptation of the produced intramedullary rod spacer to the removed intramedullary rod is what is primarily described here. However, it is of course also possible to produce a spacer with a different shape. In particular, the device is intended for single use.

In one embodiment, the device further comprises a fixing apparatus for fixing an angle between the first tube and the second tube. This allows the set angle to be fixed during casting. Any set angle can be fixed at least approximately. This effectively prevents any unintended changes in the angle. Handling during casting is facilitated, and particularly precise replicas of the explanted intramedullary rod can be produced.

The fixing apparatus may, for example, be configured to hold the first tube, the second tube and/or the connecting piece and thus prevent a change in the angle between the tubes.

The fixing apparatus may, for example, comprise a perforated plate and one or more pins. The tubes or the connecting piece can be moved to the desired angle and then placed on the plate. By inserting the pin or pins into the provided holes, the angle can then be fixed by blocking the relative rotation of the tubes against one another. The holes can, for example, be arranged on one or more circular arcs. Distances between the holes can be selected so that different angles can be set-for example, in 5° increments, for example between 0° and 40°. The plate can also have one or more support elements, which can be designed as fixed or movable pins, to fix one tube, both tubes and/or the connecting piece on one side.

In one embodiment, the device has a housing lower part which comprises a first housing lower part portion and a second housing lower part portion. A relative rotation of the two housing lower part portions is possible about an axis of rotation. In particular, the first housing lower part portion is configured to hold the first tube and/or the second housing lower part portion is configured to hold the second tube. A blocking apparatus may be provided to selectively block rotation of the two housing lower part portions.

Each housing lower part portion holds a tube. The two housing lower part portions can be rotated relative to one another about the axis of rotation to set a desired angle between the tubes. The blocking apparatus serves as a fixing apparatus. The angle between the first tube and the second tube is achieved by blocking the rotation between the two housing lower part portions.

Holding means blocking a movement in at least one movement direction. In particular, the holding apparatus blocks axial withdrawal of the first and/or second tube. In addition, the holding apparatus can block the movement of the relevant tube perpendicular to the axis and/or a rotation about a longitudinal axis and/or an axis oriented perpendicular to the longitudinal axis.

In particular, the first housing lower part portion is also configured to hold one side of the connecting piece and/or the second housing lower part portion is also configured to hold another side of the connecting piece. In particular, the first housing lower part portion comprises a receptacle for the first tube and the side of the connecting piece facing the first tube. In particular, the second housing lower part portion comprises a receptacle for the second tube and the side of the connecting piece facing the second tube.

Each housing lower part portion may comprise a disk in its central region, wherein the two disks may lie flat on one another and may be rotatable relative to one another.

The housing lower part may be configured such that the first tube, the second tube, the connecting piece and/or any combination of these components can be inserted, placed or plugged into the housing lower part in a holding position. The housing lower part can have corresponding receptacles for the first tube, the second tube and the connecting piece, in which the relevant components can be positioned. In a simple case, a receptacle can be formed by a surface with two or more pins or projections, between which the relevant tube or connecting piece can be placed against the surface. A receptacle can also be adapted to the external shape of the tube or connecting piece.

The axis of rotation is, in particular, a virtual axis of rotation. In other words, a physical axis of rotation is not necessary. The axis of rotation runs through the connecting piece.

The housing lower part can be located at the bottom when used as intended and is therefore referred to as the housing lower part. However, this is not necessary; the device can be used as intended in any orientation.

The blocking apparatus can, for example, fix a first housing lower part portion and a second housing lower part portion relative to one another, for example by way of a frictional connection and/or form-fit connection. The blocking apparatus may, for example, comprise a pin which is inserted into corresponding openings. For example, one or more openings may be present in the first housing lower part portion and in the second housing lower part portion, which openings can be aligned with one another by relative rotation of the housing lower part portions. The pin can then be inserted through an opening in each housing lower part portion to block relative rotation.

Alternatively or additionally, the blocking apparatus may be a screw, in particular one that can be turned manually, which, when screwed in, fixes the two housing lower part portions to one another. In one embodiment, the screw can be screwed into a thread of an external housing lower part portion and press onto an internal housing lower part portion. This allows for a frictional fixation. If there is also a recess in the housing lower part portion on the inside, a form-fitting connection can be created between the screw and the recess.

In one embodiment, the device has a housing upper part which comprises a first housing upper part portion and a second housing upper part portion. A relative rotation of the two housing upper part portions about the axis of rotation is possible. In particular, the housing upper part is connected and/or can be connected to the housing lower part to jointly form a housing. A blocking apparatus may be provided to selectively block rotation of the two housing upper part portions.

The first housing upper part portion may contribute to holding the first tube and/or the second housing upper part portion may contribute to holding the second tube; the first and second housing upper part portions may contribute to holding the connecting piece. For example, there may be corresponding recesses in which the relevant tube or connecting piece can be located.

The housing upper part can be designed in such a way that it forms an at least partially closed, in particular compact, housing with the housing lower part. The housing lower part and the housing upper part can be connected to one another, e.g. by pushing them together. When connected, rotation of the housing lower part and the housing upper part against one another may be blocked. In particular, the housing upper part can be removed manually and/or non-destructively from the housing lower part.

The housing then typically has a first opening for the first tube and a second opening for the second tube. In particular, the housing surrounds the connecting piece and/or protrudes beyond the connecting piece in all directions. The first housing lower part portion with the first housing upper part portion can then rotate relative to the second housing lower part portion with the second housing upper part portion. This then results in a relative rotation of the first tube in relation to the second tube.

Each housing upper part portion may comprise a disk in its central region, wherein the two disks may lie flat on one another and may be rotatable relative to one another.

In particular, the housing lower part and the housing upper part comprise corresponding shaped elements in order to enable a connection of the first housing upper part portion to the first housing lower part portion and/or a connection of the second housing upper part portion to the second housing lower part portion. Corresponding shaped elements can, for example, be projections, e.g., pins, on the one hand, and recesses, e.g., blind holes, on the other hand. Shaped elements can be designed in such a way that the housing lower part and the housing upper part can be pushed together. The plugging movement is parallel to the axis of rotation.

In one embodiment, the first tube and/or the second tube has an outwardly projecting form-fit element. In particular, the form-fit element is held in such a way that axial withdrawal of the relevant tube is blocked. In particular, the device comprises a second element which blocks the form-fit element.

The form-fit element cooperates in particular with a housing or a part thereof. For example, a housing lower part and/or a housing upper part can have an element which cooperates with the form-fit element and contacts the form-fit element and thus prevents it from being pulled out. For example, there may be a recess to accommodate the form-fit element. In particular, the form-fit element protrudes radially outwards.

In particular, the way that the tube is held by the form-fit element can be reversed. This means that, for example, after casting, the tubes and the connecting piece can be easily removed, for example after removing the housing upper part from the housing lower part.

The form-fit element can be arranged circumferentially around the first tube and/or the second tube. The form-fit element can be designed, for example, as a circumferential bead, as a circumferential projection or as a circumferential disk aligned perpendicular to the longitudinal axis. The form-fit element may have a surface oriented perpendicularly to the longitudinal axis to block withdrawal.

In particular, the connecting piece also has at least one outwardly projecting form-fit element which is held in such a way that axial withdrawal of the relevant tube is blocked. This form-fit element can also be held by a housing or a part thereof. The form-fit element of the connecting piece can be designed in the same way as the form-fit element of the first and/or second tube. In one exemplary embodiment, the connecting piece comprises two form-fit elements, wherein a first form-fit element is held by the first housing upper part portion and/or the first housing lower part portion and a second form-fit element is held by the second housing upper part portion and/or the second housing lower part portion. This prevents the connection between the connecting piece and each of the tubes from coming loose, even if the injection pressure of the casting material is increased.

In one embodiment, a length of the first tube and/or a length of the second tube is adjustable. In this way, the intramedullary rod spacer can also be adapted to the removed intramedullary rod and thus to the patient's anatomy regarding the extension of the relevant portion(s). In this way, a particularly accurate replica of the removed intramedullary rod can be achieved. There are basically a variety of options available for adjusting the length of each tube.

In one embodiment, the first tube and/or the second tube is designed for telescopic length adjustment. The length of each tube can be adjusted telescopically in this way. In particular, the tube in question comprises an outer tube with a larger first cross-section or diameter and a separate inner tube with a smaller second cross-section or diameter, which are aligned along a common longitudinal axis and connected to one another. The first or second tube in question is therefore a composite or multi-part tube. Relative movement between the inner tube and the outer tube along the longitudinal axis can occur to adjust the overall length. In other words, the inner tube can be moved within the outer tube. In particular, the outer tube is connected to the connecting piece and/or the inner tube is facing away from the connecting piece. This means that both tube parts can be pulled out together or individually after curing. Any closure elements or other components may be arranged on the tube facing away from the connecting piece.

In particular, the device further comprises a fastening apparatus for fastening the inner tube to the outer tube in a desired position. This can prevent unintended changes in length. The fastening apparatus may, for example, comprise a screw that can be screwed into a thread on the outer tube and contacts the inner tube with a frontal contact region. The screw can be aligned perpendicular to the longitudinal axis of the relevant tube. This allows a frictional connection to be created. The inner tube may have shaped elements on its outer wall that can engage with the contact region. This creates a form-fit connection to prevent unintended withdrawal.

In one embodiment, the first tube and/or the second tube can be shortened to adjust the length. In particular, the device has a cap element to be arranged on the shortened side.

The shortening is carried out in particular by means of a separation process, such as cutting or sawing. This means that an unneeded part of the tube is separated. The cap element is designed to conceal an end-face cut surface of the tube in question created by the separation. In particular, the cap element extends at least 2 cm in the axial direction of the tube in question, so that a precisely perpendicular cut is not necessary and/or any inaccuracies are concealed. This makes it easier for the user to use.

The user can then, for example, cut one or both tubes intraoperatively at the required length position(s) and cover the cut surfaces with the relevant cap elements. The cap elements can be designed for frictional connection, form-fitting connection and/or integral connection to the tube in question. The cap element can be attached and/or screwed onto the shortened tube.

The cap element can serve as an adapter for arranging one or more additional parts. The cap element may have a fastening region on the side facing away from the tube, which is designed, for example, as a thread or as part of a bayonet connection. For example, a closure element can be attached to the fastening region to close off the tube.

In one embodiment, the first tube and/or the second tube has shaped elements on its outer side for producing a latching connection. The shaped elements are arranged at intervals along the longitudinal axis of the tube. In particular, the cap element has one or more latching elements that can be latched to the relevant shaped elements. Such a cap element can also be called a locking sleeve.

Each latching element can be latched with one or more shaped elements, which are arranged in particular at the same axial position. This means that after the tube has been shortened, the cap element can be pushed onto the shortened tube and latched into place, preventing unintended removal of the cap element, particularly through a form-fit connection.

The latching elements can be hook-shaped and/or have a latching surface that is, for example, vertical or undercut in relation to the longitudinal axis of the tube in question. The shaped elements can be hook-shaped and/or have a vertical or undercut latching surface, for example. An undercut latching surface means a latching surface that is inclined in such a way that when an axial force occurs that tries to pull the tube apart, self-locking occurs. An undercut latching surface typically has an angle greater than 90° to the longitudinal axis.

The latching elements can be designed as circumferential grooves. In this case, the latching connection can be made regardless of the relative position between the tube in question and the cap element. The circumferential grooves can be sawtooth-shaped, for example with a vertical and an inclined surface.

In one embodiment, the cap element comprises a clamping part with a clamping region for clamping the shortened tube, wherein the clamping part has a first thread, e.g., an external thread. In particular, the tube can be inserted into the clamping region. In one embodiment, the cap element comprises a union nut with a second thread, e.g., an internal thread, that can be placed onto the tube. In particular, the shortened tube can be clamped by screwing the union nut onto the clamping part or by connecting the second thread and the first thread in the clamping region.

This enables a simple, frictional connection between the cap element and the tube. Any length can be set.

The clamping region may comprise circumferentially distributed, possibly tooth-like, flexible clamping elements which can be flexibly moved radially outwards or inwards when the tube is inserted into the clamping region. The clamping elements may have been made by axial slots. The end portion of the tube is then located between the clamping parts and a part of the cap element lying opposite. By screwing on the union nut, the clamping elements and the opposite part can be pressed together to clamp the tube in the clamping region.

In one embodiment, the device comprises a first end piece for arrangement at an end of the first tube or the second tube facing away from the connecting piece. In particular, a connecting region for connecting to a casting material source is arranged on a side of the first end piece facing away from the first tube or the second tube.

In one embodiment, the device comprises a second end piece for arrangement at an end of the first tube or the second tube facing away from the connecting piece. In particular, the second end piece partially closes off the tube. A ventilation opening is arranged in the second end piece, which connects the tube to the environment.

The first end piece serves primarily as an adapter for connecting to a casting material source. The first end piece is typically permeable in the axial direction. The casting material from the casting material source can be fed into the device through the first end piece to produce a spacer. The casting material source can, for example, be a cartridge in which bone cement has been produced. A casting material source can be connected directly or with an intermediate tube or hose. In particular, the casting material is pressed from the casting material source into the device. The connecting region can be designed, for example, as an internal thread or external thread. A hose or tube from the cartridge can also simply be inserted into a corresponding opening in the end piece.

The first end piece and/or the second end piece can in particular be closed off after the filling process has been completed. For example, a closure cap or a plug can be arranged on the connecting region or on another region of the first end piece. This prevents overhanging sprue or leaking casting material and ensures a clean end of the intramedullary rod spacer. The second end piece may also have a connecting region for this purpose.

In one embodiment, the device comprises a closure element for closing off the first tube, the second tube, the first end piece, the second end piece and/or a cap element. This allows the relevant tube to be closed off on the side facing away from the connecting piece. The closure element can be a screw plug with an external thread or a screw cap with an internal thread.

A closure element can also be designed as a plug, for example made of plastic or elastomer. The part receiving the plug and/or the plug may be conically tapered to facilitate pressing in.

The second end piece serves in particular as a closure and/or vent. When casting material is added to the device, the displaced air must escape from the device. The ventilation opening is provided for this purpose. In particular, the ventilation opening has a cross-section or diameter of one or several millimeters.

The first end piece and/or the second end piece can be arranged directly or indirectly on the relevant tube. In an indirect arrangement, one or more intermediate pieces may be present. For example, a cap element as described above can be arranged as an intermediate piece between the tube and the end piece, even if shortening of the tube is not intended.

Alternatively, the cap element can function as the first or second end piece. The first end piece and/or the second end piece can be made of one or more parts.

The first end piece and/or the second end piece may have a passage for an inlay. An inlay can be passed through the end piece, for example, to be held in place in this way. Partial passage is also possible; in this case the inlay ends in the passage. When connected, the passage can be partially or completely closed off or sealed by the inlay. The passage can be adapted to the inlay in terms of its extension, particularly its cross-section.

The passage may comprise at least one recess, for example in the form of a groove, wherein the recess extends in the axial direction through a thread of a connecting region of the first or second tube. If the inlay is in the passage, another component with a corresponding thread can be screwed onto the thread even if the inlay is present, without shear forces acting on the inlay. If a plug is provided, the part receiving the plug can also run in an axial direction through the connecting region. If there are multiple recesses or grooves, for example distributed in the circumferential direction, threading is easier.

In particular, the device is designed such that an inner cross-section or diameter in the region of the first tube adjacent to the connecting piece corresponds at least approximately to the inner cross-section of the adjacent portion of the connecting piece and/or that an inner cross-section or diameter in the region of the second tube adjacent to the connecting piece corresponds at least approximately to the inner cross-section of the adjacent portion of the connecting piece. In this way, a substantially constant outer cross-section or diameter of the intramedullary rod spacer can be produced.

In one embodiment, the connecting piece has a first connecting region for plugging onto the first tube, a second connecting region for plugging onto the second tube and a central region located between the connecting regions. In particular, an inner cross-section of the connecting piece is smaller in the central region than in the first and second connecting regions.

The connecting piece is designed as a sleeve, i.e., as a connecting piece arranged on the outside of the tube. The connecting piece can be pushed onto the first tube and/or the second tube. In particular, the connecting piece can then be removed from the first tube and/or the second tube.

The connecting piece may be made of a flexible material, an elastomer and/or a silicone, for example a biocompatible rubber-elastic plastics material, such as silicone rubber or EPDM rubber. In particular, the connecting piece is made of a material with a Shore A hardness equal to or greater than 50.

In particular, the connecting piece is so flexible that in the relevant connecting region it has, at least in some regions, a smaller inner diameter than the outer diameter of the corresponding tube. The connecting piece can then be frictionally fastened to the tube, due to a radially inwardly directed restoring force. In this way, leakage of casting material between the connecting piece and the tube can be prevented, even if the casting material is subjected to elevated injection pressure. In addition, unintended loosening can be prevented.

In particular, the first tube and/or the second tube is made of a material with greater rigidity and/or strength than the connecting piece and/or of a thermoplastic material.

In one embodiment, the first tube, the second tube and/or the connecting piece are made of a translucent and/or transparent material. This allows the filling process to be visually monitored.

In particular, the difference in the inner diameters corresponds to twice the wall thickness of the relevant tube. In this way, a continuous outer surface of the spacer without cracks can be created even in the transition region between the connecting piece and the relevant tube.

Alternatively, the connecting piece may also have connecting regions that are designed to be inserted into the relevant tube. In this case, the relevant tube may have an enlarged inner diameter in the connecting region to enable a constant inner surface of the connection.

In one embodiment, the device comprises an inlay arranged in the first tube and/or the second tube for internally reinforcing the intramedullary rod spacer being produced.

The inlay is made primarily of metal and can also be referred to as a metal core. The inlay serves to mechanically reinforce the intramedullary rod spacer. In particular, the inlay is positioned approximately centrally in the tube in question to then lie approximately centrally in the spacer. The inlay can be held in the tube in question using suitable holders. In particular, at least three holders or spacers are provided in the circumferential direction to keep the inlay approximately central. Spacers may be attached to the inlay. The inlay can be positioned exactly in the middle. However, it is acceptable if the inlay is located in a central region corresponding to half the inner radius of the tube in question.

The inlay may extend over part of the length of the first and/or second tube or over the entire length. An inlay/the inlay can also be arranged in the connecting piece. In one embodiment, a continuous inlay is arranged that extends from the first tube via the connecting piece into the second tube. Kirschner wire, for example, can be used as an inlay.

In principle, mechanical stabilization of the spacer is not always necessary because, due to the severity of the disease, mobilization of the patient is not possible in many cases. In this case, no inlays are required. However, it may be planned to provide an inlay for all spacers as standard. This can reduce complexity, as just one device can be used for all applications.

A further aspect of the invention is a kit, comprising:
a device according to the invention, and
at least one further first tube, wherein the further first tube has a different length, a different shape and/or a different cross-section, in particular diameter, than the first tube of the device, wherein the further first tube can be connected to the connecting piece instead of the first tube, or
at least one further second tube, wherein the further second tube has a different length, a different shape and/or a different cross-section, in particular diameter, than the second tube of the device, wherein the further second tube can be connected to the connecting piece instead of the second tube.

The kit therefore contains multiple first and/or second tubes so that the appropriate tube can be selected that is best suited to replicate the explanted intramedullary rod.

A further aspect of the invention is the use of a casting mold for producing an intramedullary rod spacer. A device according to the invention is used as a casting mold. All features, advantages and configurations of the above-mentioned device also apply to the use and vice versa. Polymethylmethacrylate bone cement is added to the casting mold.

A further aspect of the invention is a method for producing an intramedullary rod spacer using a device, wherein the device comprises a first tube, a second tube and a flexible connecting piece for fluidically connecting the first tube to the second tube. The method comprises positioning the first tube and the second tube at a desired angle relative to one another, as well as filling a casting material into a first tube, the second tube and the connecting piece to produce the intramedullary rod spacer. Filling is carried out in such a way that the entire interior of the device, i.e., both tubes and the connecting piece is filled. Filling can be done by pressing. All features, advantages and configurations of the above-mentioned device also apply to the method and vice versa. In particular, the casting material comprises polymethylmethacrylate bone cement and optionally one or more medicinal active ingredients.

The method may further comprise one or more of the following steps, in any combination:
adjusting the length of the first tube and/or the second tube, for example by shortening the tube in question and arranging a cap element and/or by telescopic length adjustment, and optionally locking the adjusted length,
fixing the set angle using a fixing apparatus,
connecting a tube to a casting material source, for example by means of a first end piece,
after filling: removing the casting material source and optionally closing off the tube or the component on which the casting material source was arranged,
removing excess casting material from the end of the device where the casting material source was not located and/or closing off the component located at that end,
curing the casting material,
demolding the produced spacer, for example by pulling the first and/or second tube off the spacer and optionally cutting open the connecting piece and/or pulling the connecting piece off the spacer.
removing the spacer.

### BRIEF DESCRIPTION OF THE DRAWINGS

Exemplary embodiments of the invention are also explained in greater detail below with reference to figures. Features of the exemplary embodiments can be combined individually or in a plurality of the claimed subjects, unless otherwise indicated. The claimed scope of protection is not limited to the exemplary embodiments.

### IN THE DRAWINGS

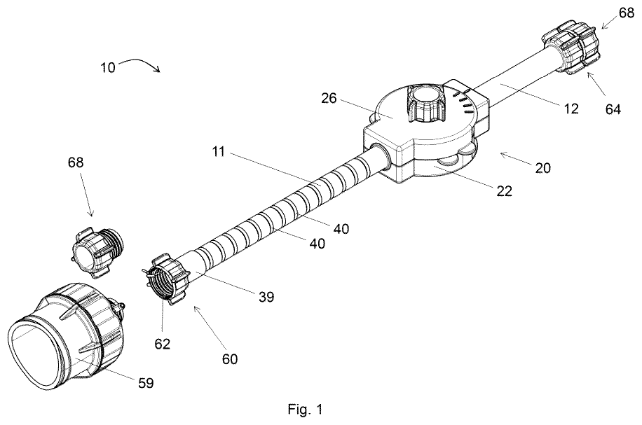

FIG. 1 is a perspective view of a device;

FIG. 2 is an exploded perspective view of a device;

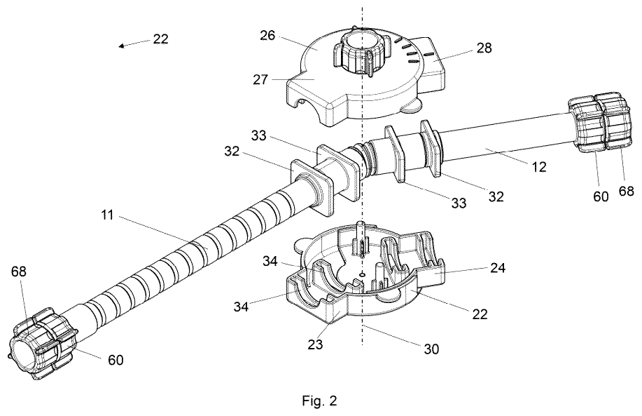

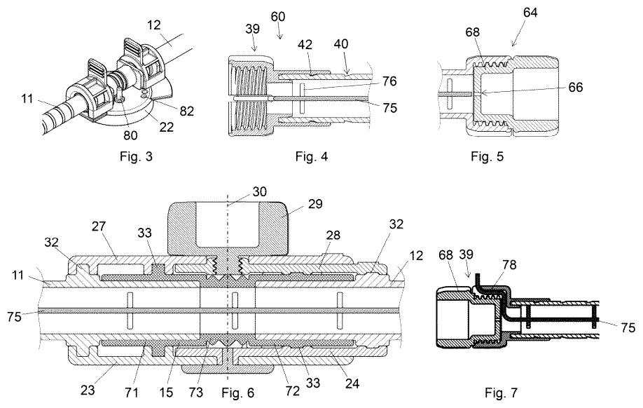

FIGS. 3 to 7 are enlarged detail views;

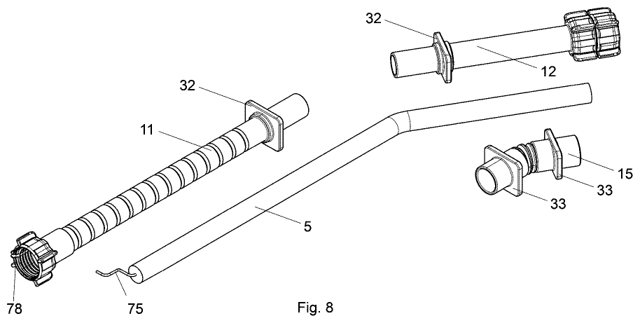

FIG. 8 shows parts of a device together with a produced spacer; and,

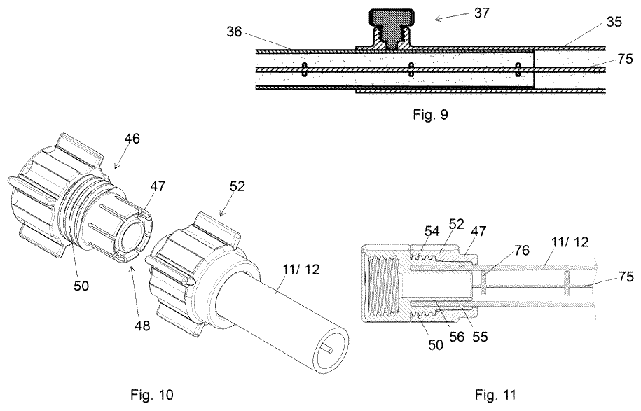

FIGS. 9 to 11 are enlarged detail views.

### DETAILED DESCRIPTION OF THE INVENTION

FIG. 1 shows a device 10 according to the invention for producing an intramedullary rod spacer. The device 10 comprises a first tube 11, a second tube 12 and a flexible connecting piece which fluidically connects the first tube 11 to the second tube 12. The connecting piece is arranged in a housing 20, which comprises a housing lower part 22 and a housing upper part 26. At the free end of the first tube 11 shown at the bottom left, a first end piece 60 is arranged, which has a connecting region 62 with an internal thread for mechanical connection to a casting material source 59. The casting material source 59 is, for example, a cartridge with bone cement, which is partially shown and which has a dispensing tip with an external thread. The external thread can be connected to the internal thread of the connecting piece 62 in order to press the casting material through the first tube 11 into the entire interior of the device. In other words, the device 10 can be used as a casting mold to produce an intramedullary rod spacer from a casting material, e.g., bone cement.

The device 10 further comprises a closure cap 68 which can be connected to the connecting region 62 after the casting material has been filled. At the free end of the second tube 12 shown at the top right, a second end piece 64 is arranged, which also comprises a closure cap 68.

The first tube 11 is typically a distal tube in which a distal portion of the intramedullary rod spacer is produced. This is usually longer than the proximal second tube 12. Typically, according to the intramedullary rod to be replicated, the second tube 12 has an inner diameter that corresponds at least to the inner diameter of the first tube 11.

The first tube 11 is adjustable in length. This is addressed, for example, in such a way that the first tube 11 has shaped elements 40 along its longitudinal direction, typically at regular intervals, and a cap element 39 has corresponding latching elements 42. The first tube 11 can therefore be shortened at any desired position, for example by cutting. The cap element 39 can then be plugged onto it, wherein one or more latching elements 42 of the cap element 39 latch with one or more shaped elements 40 of the first tube 11. In this way, axial removal of the cap element 39 is prevented by a form-fit connection. This is shown as an enlarged detail in FIG. 4, which additionally shows an inlay 75 located centrally in the first tube 11, and also spacers 76 for holding the inlay 75 in the central position.

FIG. 2 shows an illustration similar to FIG. 1, in which the individual parts of the housing 20 are shown in an exploded view. The connecting piece 15 located centrally between the first tube 11 and the second tube 12 is visible, and fluidically connects the interior spaces of the two tubes 11, 12. The connecting piece 15 also has a tubular basic shape and is made of flexible, for example rubber-elastic, material. As such, any angle between the first tube 11 and the second tube 12 can be set within a predetermined angle range of approximately 0° to 40°.

In their corresponding regions facing the connecting piece 15, both the first tube 11 and the second tube 12 have a form-fit element 32 in the form of a circumferential disk. The connecting piece 15 also has such a form-fit element 33 on each of its sides.

It can be seen that the housing lower part 22 comprises a first housing lower part portion 23 which receives the first tube 11 and a second housing lower part portion 24 which receives the second tube 12. The first housing lower part portion 23 and the second housing lower part portion 24 are rotatable relative to one another about a common axis of rotation 30. Analogously, the housing upper part 26 comprises a first housing upper part portion 27 and a second housing upper part portion 28 which is rotatable relative thereto about the same rotation axis 30.

The housing lower part 22 and the housing upper part 26 can typically be connected to one another or, as shown, separated from one another depending on requirements. This is preferably done manually and non-destructively. In the example shown here, the housing lower part 22 comprises upwardly pointing pins which can be inserted into corresponding openings in the housing upper part 26 in order to close off the housing 20. However, any other closure options are possible.

Recesses 34 for receiving the form-fit elements 32, 33 are provided in the housing lower part 22 and in the housing upper part 26, respectively. If the unit comprising the first tube 11, the connecting piece 15 and the second tube 12 is placed in the housing lower part 22 and closed off with the housing upper part 26, neither the connection between the first tube 11 and the connecting piece 15 nor the connection between the connecting piece 15 and the second tube 12 can be released.

FIG. 6 shows a sectional view of such a connection between the first tube 11, the connecting piece 15 and the second tube 12. Here it is clearly visible that, on the left side, the first housing lower part portion 23 and the first housing upper part portion 27 hold both the first tube 11 (see form-fit elements 32) and the left part of the connecting piece 15 (see form-fit elements 33). On the right side, however, the right part of the connecting piece 15 and the second tube 12 are held by the second housing upper part portion 28 and the second housing lower part portion 24. Furthermore, the second housing lower part portion 24 and the second housing upper part portion 28 are surrounded on the right side by the first housing lower part portion 23 and the first housing upper part portion 27. During a relative rotation, the first housing upper part portion 27 slides relative to the second housing upper part portion 28 and the first housing lower part portion 20 slides relative to the second housing lower part portion 24.

In addition, a blocking apparatus 29 is shown, with which the rotation of the two housing lower part portions 23, 24 as well as the rotation of the two housing upper part portions 27, 28 can be blocked. The blocking apparatus 29 is designed in this case by way of example as a screw aligned along the rotation axis 30, which can be screwed into a thread in the second housing upper part portion 28 and subsequently clamps the first housing upper part portion 27 between the second housing upper part portion 28 and the head of the screw, so that sliding is no longer possible and the angle between the first tube 11 and the second tube 12 is consequently fixed.

FIG. 6 further shows that the connecting piece 15 has a first connecting region 71 for plugging onto the first tube 11, a second connecting region 72 for plugging onto the second tube 12 and a central region 73 located therebetween. The inner diameter of the connecting piece 15 is smaller in the central region 73 than in the first and second connecting regions 71, 72, in such a way that the central region 73 has the same inner diameter as the adjacent region of the first tube 11 and the second tube 12.

FIG. 3 shows an alternative way to fix the angle, i.e. an alternative blocking apparatus. In this case, openings 82 are arranged in the first housing lower part portion and in the second housing lower part portion, and there is at least one pin like the pin 80 shown which can be inserted into the openings. The pin thus passes through the opening in one of the housing lower part portions and into the opening in the housing lower part portion below it, so that relative rotation of the two housing lower part portions is no longer possible.

FIG. 5 shows an enlarged detail of the second end piece 64, in which it can be seen that in the closure cap 68 there is a radially and centrally arranged ventilation opening 66. This allows the escape of air displaced during the pressing-in of the casting material.

FIG. 7 shows an enlarged detail, for example of the first end piece, in which it can be seen how a metallic inlay 75 can be held in the region of a cap element 39. In the region of the thread shown above, for example the connecting region 62, there is a recess 78 in the form of a groove running parallel to the longitudinal axis of the tube in question. The inlay 75, for example a Kirschner wire, is then placed in the recess 78 before screwing in the closure cap 68, and then the closure cap 68 is screwed on.

FIG. 8 shows an overview of the first tube 11, the second tube 12, the connecting piece 15 and a produced intramedullary rod spacer 5 after demolding. After removing the housing, both the first tube 11 and the second tube 12 can typically be pulled outwards. The connecting piece 15 can then also be pulled off or removed, for example by cutting it open. The resulting intramedullary rod spacer 5 with the previously set angle is now completed.

FIG. 9 shows a further detail in which at least one of the tubes 11, 12 is telescopically extendable. The relevant tube comprises an outer tube 35 and an inner tube 36, which is axially displaceable in the outer tube 35. Preferably, a fastening apparatus 37 is provided for fastening the outer tube 35 to the inner tube 36. In the example shown here, this comprises a screw which can be screwed into a thread on the outer tube 35, for example perpendicular to the longitudinal axis of the tube, and which subsequently presses onto the inner tube 36 from the outside in order to create a frictional connection.

FIGS. 10 and 11 show enlarged details of another alternative for adjusting the length of one or both tubes 11, 12. The cap element 39 here comprises a clamping part 46 shown on the left with a clamping region 48 into which the tube 11 or 12 can be inserted in the axial direction. The clamping region 48 is defined by flexible clamping elements 47, which are located here on the outside, as an example, and an internal, opposite part 56. The clamping elements 47 are produced, for example, by axially slitting an outer, tubular portion of the clamping part 46. The opposite part 56 is, for example, an inner, tubular portion of the clamping part 46. The clamping part 46 further comprises a first thread 50.

The cap element 39 further comprises a union nut 52 which is pushed over the relevant tube 11 or 12 and comprises a second thread 54 corresponding to the first thread 50. If the union nut 52 is then screwed onto the clamping part 46, the clamping elements 47 are pressed axially inwards and thus clamp the relevant tube 11 or 12 against the opposite part 56. Consequently, the cap element 39 is connected to the tube 11 or 12 with a frictional connection. In addition, radially-inwardly-projecting clamping projections 55 may be present on one or more clamping elements 57, as shown in FIG. 11, which, depending on the design and properties of the materials, enable a frictional connection and/or form-fitting connection. Alternatively, or additionally, the clamping region 48 can be conical in order to enable and/or improve the connection.

### LIST OF REFERENCE SIGNS

Intramedullary rod spacer 5
Device 10
First tube 11
Second tube 12
Connecting piece 15
Fixing apparatus 18
Housing 20
Housing lower part 22
First housing lower part portion 23
Second housing lower part portion 24
Housing upper part 26
First housing upper part portion 27
Second housing upper part portion 28
Blocking apparatus 29
Axis of rotation 30
Form-fit element 32
Form-fit element 33
Recess 34
Outer tube 35
Inner tube 36
Fastening apparatus 37
Cap element 39
Shaped elements 40
Latching elements 42
Clamping part 46
Clamping element 47
Clamping region 48
First thread 50
Union nut 52
Second thread 54
Clamping projection 55
Opposite part 56
Casting material source 59
First end piece 60
Connecting region 62
Second end piece 64
Ventilation opening 66
Closure cap 68
First connecting region 71
Second connecting region 72
Central region 73
Inlay 75
Spacer 76
Recess 78
Pin 80
Opening 82

What is claimed is:

1. A device for producing an intramedullary rod spacer, comprising a first tube, a second tube, and a flexible connecting piece for fluidically connecting the first tube to the second tube, wherein the first tube and the second tube can be positioned at different angles relative to one another.

2. The device according to claim 1, wherein the device further comprises a fixing apparatus for fixing an angle between the first tube and the second tube.

3. The device according to claim 2, wherein the device comprises a housing lower part which comprises a first housing lower part portion and a second housing lower part portion, wherein a relative rotation of the two housing lower part portions about an axis of rotation is possible, wherein the first housing lower part portion is designed to hold the first tube and the second housing lower part portion is designed to hold the second tube, wherein in particular a blocking apparatus is provided in order to selectively block a rotation of the two housing lower part portions.

4. The device according to claim 3, wherein the device comprises a housing upper part which comprises a first housing upper part portion and a second housing upper part portion, wherein a relative rotation of the two housing upper part portions about the axis of rotation is possible, wherein the housing upper part is connected or can be connected to the housing lower part in order to jointly form a housing, wherein in particular a blocking apparatus is provided in order to selectively block a rotation of the two housing upper part portions.

5. The device according to claim 2, wherein the first tube and/or the second tube comprises an outwardly projecting form-fit element which is held in such a way that axial withdrawal of the relevant tube is blocked.

6. The device according to claim 1, wherein a length of the first tube and/or a length of the second tube is adjustable.

7. The device according to claim 1, wherein the first tube and/or the second tube is designed for telescopic length adjustment.

8. The device according to claim 6, wherein the first tube and/or the second tube can be shortened to adjust the length, and the device has a cap element for arrangement on the shortened side.

9. The device according to claim 1, wherein the first tube and/or the second tube comprises, on its outer side, shaped elements for producing a latching connection, and these are arranged at intervals along the longitudinal axis of the tube, and the cap element comprises one or more latching elements which can be latched to the relevant shaped elements.

10. The device according to claim 8, wherein the cap element comprises:
a clamping part with a clamping region for clamping the shortened tube, wherein the clamping part comprises a first thread; and,
a union nut with a second thread which can be pushed onto the tube, wherein the shortened tube can be clamped in the clamping region by screwing the union nut onto the clamping part.

11. The device according to claim 1, further comprising:
a first end piece for arrangement at an end of the first tube or of the second tube facing away from the connecting piece, wherein a connecting region for connection to a casting material source is arranged on a side of the first end piece facing away from the first tube or the second tube; and/or,
a second end piece for arrangement at an end of the first tube or of the second tube facing away from the connecting piece, wherein a ventilation opening is arranged in the second end piece and connects the tube to the environment.

12. The device according to claim 1, wherein the connecting piece has a first connecting region for being pushed onto the first tube, a second connecting region for being pushed onto the second tube and a central region located between the connecting regions, wherein an inner cross-section of the connecting piece is smaller in the central region than in the first and second connecting regions.

13. The device according to claim 1, further comprising an inlay arranged in the first tube and/or the second tube for internally reinforcing the intramedullary rod spacer being produced.

14. A use of a casting mold to produce an intramedullary rod spacer.

15. A method for producing an intramedullary rod spacer using a device, wherein the device comprises a first tube, a second tube and a flexible connecting piece for fluidically connecting the first tube to the second tube, wherein the method comprises:
positioning the first tube and the second tube at a desired angle relative to one another; and,
filling a casting material into a first tube, the second tube and the connecting piece to produce the intramedullary rod spacer.

16. The device according to claim 3, wherein the first tube and/or the second tube comprises an outwardly projecting form-fit element which is held in such a way that axial withdrawal of the relevant tube is blocked.

17. The device according to claim 4, wherein the first tube and/or the second tube comprises an outwardly projecting form-fit element which is held in such a way that axial withdrawal of the relevant tube is blocked.

18. The device according to claim 7, wherein the first tube and/or the second tube can be shortened to adjust the length, and the device has a cap element for arrangement on the shortened side.

19. The device according to claim 9, wherein the cap element comprises:
a clamping part with a clamping region for clamping the shortened tube, wherein the clamping part comprises a first thread; and,
a union nut with a second thread which can be pushed onto the tube, wherein the shortened tube can be clamped in the clamping region by screwing the union nut onto the clamping part.
